(19) United States
(12) Patent Application Publication
(10) Pub. No.: US 2008/0045862 A1
(43) Pub. Date: Feb. 21, 2008

(54) SYSTEMS, METHODS, AND DEVICES FOR SAMPLING BODILY FLUID

(76) Inventors: Corey Dalebout, Lincoln, CA (US); Josh Sybrowsky, Lincoln, CA (US)

Correspondence Address: WORKMAN NYDEGGER, 60 EAST SOUTH TEMPLE, 1000 EAGLE GATE TOWER, SALT LAKE CITY, UT, 84111, US

(21) Appl. No.: 11/765,888

(22) Filed: Jun. 20, 2007

Related U.S. Application Data
(60) Provisional application No. 60/805,426, filed on Jun. 21, 2006.

(51) Int. Cl. A61B 5/157

(52) U.S. Cl. 600/573

(57) ABSTRACT

A fluid sampling system is disclosed comprising a fluid drawing device and a fluid sampling device. In one embodiment, the fluid drawing device comprises a venous arterial blood management protection apparatus having a housing with a fill chamber in fluid communication with a patient injection site, a piston positionable within the fill chamber to sealingly engage the fill chamber, wherein the piston has a first position and a second position, wherein movement of the piston from the first position to the second position generates a negative pressure in the fill chamber effective to draw fluid from the patient injection site into the fill chamber, biasing means for biasing the piston to the second position relative to the first position, and securing means for selectively securing the piston in the second position. In one embodiment, the fluid sampling device comprises a base portion having a canula, a handle, and a ridge extending around at least a portion of an outer surface; and a top portion having a flange adapted to engage said the ridge to couple the top portion to the base portion.

### CROSS-REFERENCE TO RELATED APPLICATIONS

This application claims the benefit of U.S. Provisional Application No. 60/805,426, filed Jun. 21, 2006, the contents of which is hereby incorporated by reference in its entirety.

### BACKGROUND OF THE INVENTION

1. The Field of the Invention

The invention relates to medical systems, methods, and devices, more specifically, the invention relates to a fluid sampling system.

2. Relevant Technology

In some medical procedures, the condition of a patient can require that an intravenous/intra-arterial tube or catheter be inserted into a blood vessel. The patient's blood vessel can be connected by the tube to a source of fluid which can provide fluid, such as a medicament, and which can also be connected to a pressure transducer that senses the pressure within the patient's blood vessel.

In critical care situations, it can be necessary to periodically obtain samples of the patient's bodily fluids, such as blood. For procedures carried out using a needle stick, the likelihood of a health care worker being inadvertently stuck can increase, thereby increasing the risk of infection from a contaminated needle. Rather than stick a patient with a needle each time blood must be drawn, blood can be drawn through the tube already connected to the patient's blood vessel. Since the tube connected to the patient's blood vessel can contain fluid other than blood, such as saline solution and some medication, it is useful to draw the patient's blood up into the tube so that a blood sample can be obtained which is substantially unadulterated by the fluid which is being supplied to the tube by an external source. After the substantially unadulterated blood has been drawn up the tube to a sampling site, the blood sample can be collected into a sample container.

Disadvantageously, many of the previously available devices require two-handed operation by a medical practitioner. Some of the previous devices utilize a conventional medical syringe to create the suction necessary to draw the blood up the tube. Such syringes are often unwieldy to use and their typical long, narrow dimensions makes them cumbersome. Many of the previously available devices are complicated and expensive. Moreover, some of the previously available devices include sharp bends in the fluid path and/or relatively long supplementary fluid paths both of which result in residual blood and fluid remaining in the fluid path which can cause problems such as clotting.

In 2001, a study of 1548 patients was performed to demonstrate the effects of “intensive insulin therapy” on mortality and morbidity. See Greet Van den Bergh, et. al., Intensive Insulin Therapy in Critically Ill Patients, The New England Journal of Medicine, Vol. 345:1359-1367, No. 19, Nov. 8, 2001. The study showed that patients with tightly controlled blood glucose levels (between 80-110 mg/dl) had remarkably improved outcomes. Overall mortality was decreased by 34%, blood stream infections by 46%, acute renal failure requiring dialysis or hemofiltration decreased by 41%, the median number of red cell transfusions by 50% as well as requiring less time on the ventilator and less days in the ICU. Beyond the increased health benefits to patients, this reduces hospital costs by $3000 to $4000 per ICU patient. See http://www.santacruzsentinel.com/archive/2005/February/13/biz/stories/02biz.htm.

The medical community has been striving for successful implementation of intensive insulin therapy because of the documented benefits of intensive insulin therapy. In order to implement this therapy, patients can have their fingers stuck for glucose readings every hour for days, weeks and even months. This has caused a significant amount of pain and torment to be inflicted to the patients. Additionally, repeated glucose level monitoring can take up valuable time of registered nurses (RNs) and practitioners.

### BRIEF SUMMARY OF THE INVENTION

Example embodiments of the medical system described herein can enable the user more freedom to deal with positional lines, obtain glucose readings and can save time by initiating the testing process. This system can accomplish various significant improvements over the prior art while still garnering the significant benefits of intensive insulin therapy and being adaptable to nearly all hospitals. One benefit of the present invention is that it can reduce pain and discomfort of patients by reducing repeated finger pricking and venous sticks to obtain lab samples. Further, the present invention can decrease the time necessary for practitioners to ascertain patient's glucose levels and obtain blood samples for lab use. Another benefit of the present invention is that it can decrease the risk to practitioners and patients by reducing the need for needles used in the transfer of blood from sample ports to the test strip, and those used with phlebotomy. Additionally, the present invention can decrease cross contamination risk by utilizing a contained blood sample within the sampling device.

Example embodiments the medical system of the present invention can include a medical device for drawing a patient's blood into an intravenous tube. The medical device can include a housing having a fill chamber into which a patient's blood can be drawn. The fill chamber can be in fluid communication with a patient injection site, such as a sample port of an intravenous tube. A piston can be positioned within the fill chamber. The outer edge of the piston and the interior walls of the fill chamber can be configured to substantially create a seal between the piston and the interior walls of the fill chamber. The piston can be moved from a first position to a second position, which can create a negative pressure within the fill chamber that can draw fluid from the patient injection site into the fill chamber. The present embodiment can also include biasing means, such as a spring or fluid pressure in the line or tube, for biasing the piston to the second position. The medical system can further include securing means, such as clamps, engaging ridges, and the like, for selectively securing the piston in the first and/or second positions.

The medical system can also include a medical device for sampling bodily fluid, such as blood. The fluid sampling device can comprise a base portion and a top portion. The base portion can include a blunt canula that can be inserted into a sample port of an intravenous tube or catheter. The base portion can also have at least one handle for a user to hold and which facilitates use of the medical device. Extending around at least a portion of the base portion's outer edge can be a ridge that can be configured to engage the top portion. The top portion can include a flange adapted to engage the base portion's ridge in order to couple the top portion to the base portion. In some embodiments, the top portion and the base portion are designed to couple together with a test strip movably disposed therebetween. The fluid sampling device can also include a protective housing for preventing undesired damage or contamination of the fluid sampling device.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates the fluid sampling system according to one embodiment of the present invention;

FIG. 2A illustrates a cross-sectional view of a fluid drawing device according to one embodiment of the present invention;

FIG. 2B illustrates a cross-sectional view the fluid drawing device of FIG. 2A after the fluid drawing device has been activated;

FIG. 3 illustrates a cross-sectional view of an alternate example embodiment of a fluid drawing device of the present invention;

FIG. 4A illustrates a top view of a fluid sampling device according to one embodiment of the present invention;

FIG. 4B illustrates a side view of the fluid sampling device of FIG. 4A;

FIG. 5A illustrates a top view of an alternative embodiment of the fluid sampling device of the present invention;

FIG. 5B illustrates a bottom view of the alternative embodiment of the fluid sampling device of FIG. 5A;

FIG. 5C illustrates a cross-sectional view of the alternative embodiment of the fluid sampling device of FIG. 5A;

FIG. 5D illustrates a cross-sectional side view of the alternative embodiment of the fluid sampling device of FIG. 5A;

FIG. 6 illustrates a cross-sectional side view of another alternative embodiment of the fluid sampling device of FIG. 5A;

FIG. 7 illustrates a modified glucometer according to one embodiment of the present invention;

FIG. 8 illustrates a perspective view of a fluid sampling device according to another embodiment of the present invention;

FIG. 9A illustrates an end view of the top portion of the fluid sampling device of FIG. 8;

FIG. 9B illustrates a side view of the top portion of the fluid sampling device of FIG. 8;

FIG. 9C illustrates a bottom view of the top portion of the fluid sampling device of FIG. 8;

FIG. 10A illustrates an end view of the base portion of the fluid sampling device of FIG. 8;

FIG. 10B illustrates a side view of the base portion of the fluid sampling device of FIG. 8;

FIG. 10C illustrates a top view of the base portion of the fluid sampling device of FIG. 8;

FIG. 11 illustrates a cross-sectional end view of the fluid sampling device of FIG. 8;

FIG. 12A illustrates a perspective view of the fluid sampling device of FIG. 8 disposed within a protective packaging;

FIG. 12B illustrates a partial cross-sectional view of the protective packaging of FIG. 12A.

### DETAILED DESCRIPTION OF THE PREFERRED EMBODIMENTS

Embodiments of the present invention described herein relate to a fluid sampling system. The fluid sampling system can include a fluid drawing device, a fluid sampling device, and a modified glucometer to accommodate the fluid sampling device. Other standard medical equipment used in conjunction with these elements can include a pressure transducer, an IV stand, a pressure bag, saline solution, IV tubing, and a peripheral IV, an Arterial Line or a Central Venous Line, for example.

The fluid drawing device of the fluid sampling system can be used to draw bodily fluid, such as blood, from a patient injection site into an IV tube or catheter. After the fluid has been drawn into the IV tube, the fluid sampling device can be introduced into a sample port of the IV tube to retrieve a sample of the bodily fluid. After a fluid sample has been retrieved, the fluid sample can be analyzed with a glucometer that can be modified to accommodate the fluid sampling device. The fluid sampling system of the present invention can provide a safe method of obtaining a sample of bodily fluid from a patient. For instance, the fluid sampling system can reduce the need to use needles each time a blood sample is needed, which in turn can reduce the pain and discomfort a patient experiences each time he or she is pricked. Further, the possibility that a health care worker will be pricked with a contaminated needle can be reduced with use of the fluid sampling system. Additionally, the fluid sampling system can be simple to use, thus allowing health care workers to focus on other aspect of the patient's treatment.

In one example embodiment, an IV tube is connected to a pressure bag (or a pressure transducer) at one end thereof, while the other end of the IV tube is in fluid communication with a patient injection site. Unlike typical IV systems, the present example embodiments also have a fluid drawing device connected to the IV tube between the pressure bag and the patient such that fluid flowing through the IV tube also flows through the fluid drawing device. In addition, the IV tube also includes a sample port between the fluid drawing device and the patient. The fluid sampling device can be inserted into the sample port in order to take a sample of the fluid in the IV tube.

When the fluid sampling system is configured as described above, a user, such as a doctor or nurse, can take a sample of a patient's bodily fluid, such as blood, by activating the fluid drawing device, which draws the bodily fluid into the IV tube past the sample port, allowing the user to access the bodily fluid at the sample port with the fluid sampling device. The fluid drawing device has a fill chamber through which the fluid from the IV tube can flow. Disposed within the fill chamber is a plunger that can be positioned between a first position and a second position. The plunger can be locked in the first and/or the positions. To activate the fluid drawing device, the user releases the locking mechanism on the plunger. Once the plunger is unlocked, the plunger can move toward the second position, thus creating a negative pressure in the IV tube, which draws the bodily fluid into the IV tube past the sample port. The user can then insert the fluid sampling device into the sample port to retrieve a sample of the bodily fluid. When a sufficient fluid sample has been retrieved, the fluid sampling device can be removed from the sample port and the user can depress the plunger of the fluid drawing device to force the extra bodily fluid back into the patient. The fluid sampling device can be configured to have a test strip disposed therein. When the bodily fluid enters the fluid sampling device from the sample port, the bodily fluid can be absorbed by the test strip. The sample of bodily fluid absorbed by the test strip can then be analyzed by a glucometer.

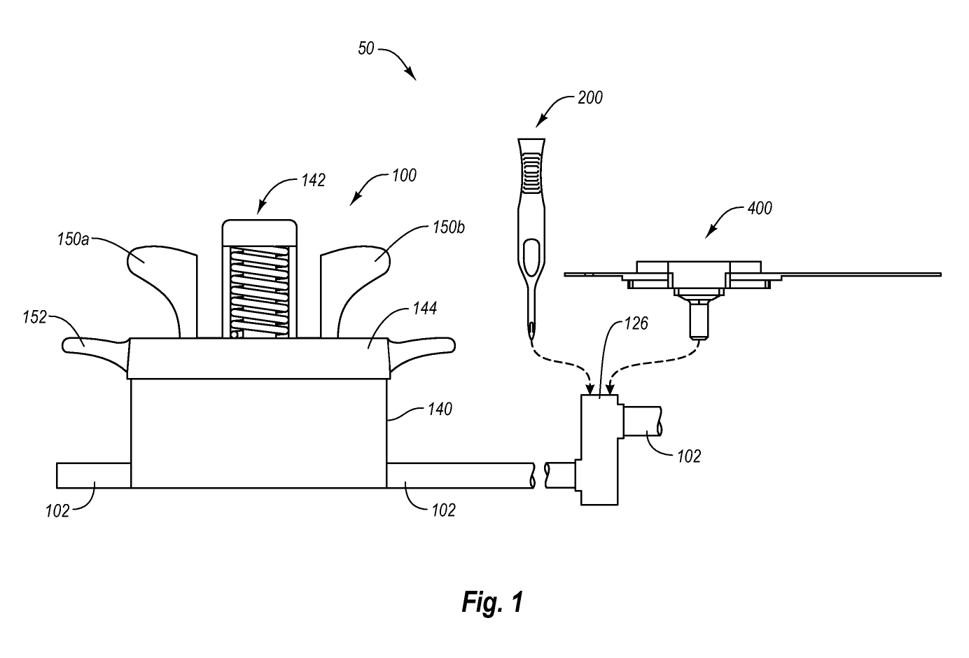

As seen in FIG. 1, an exemplary embodiment of the fluid sampling system 50 can include an IV tube 102, a fluid drawing device 100, a sample port 126, and a fluid sampling device 200 and/or 400. Fluid drawing device 100 is in fluid communication with IV tube 102 such that fluid drawing device 100 can draw fluid through IV tube 102 past sample port 126. After fluid drawing device 100 has drawn fluid past sample port 126, fluid sampling device 200 or 400 can be introduced into sample port 126 to obtain a fluid sample.

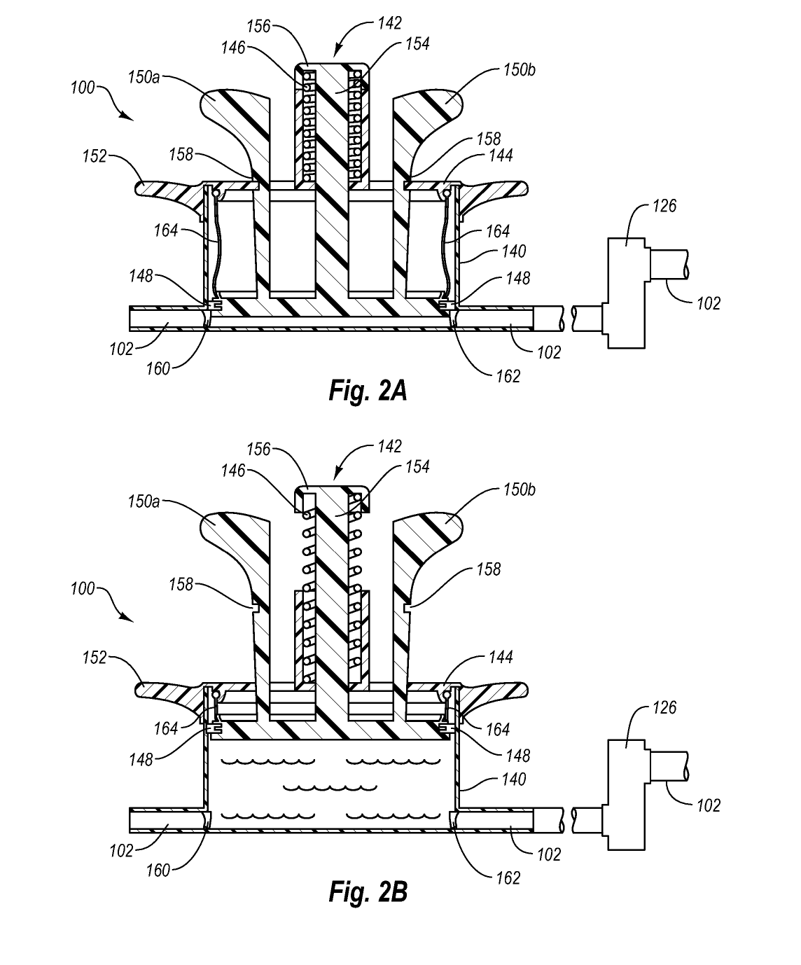

FIGS. 2A-2B illustrate an exemplary embodiment of fluid drawing device 100. In this embodiment, fluid drawing device 100 comprises a housing 140, a piston 142 partially disposed within housing 140, locking mechanism 150 connected to piston 142, a cap 144 coupled to the top of housing 140, and a spring 146. Housing 140 of fluid drawing device 100 is in fluid communication with IV tube 102 such that fluid flowing through IV tube 102 flows from a first portion of IV tube 102 through housing 140 and into a second portion of IV tube 102. At the point of connection between the first portion of IV tube 102 and housing 140 there is a first valve 160. First valve 160 is a one-way valve that allows fluid to flow from the first portion of IV tube 102 into housing 140. At the point of connection between housing 140 and the second portion of IV tube 102 there is a second valve 162. Second valve 162 is configured to allow fluid to flow both into and out of housing 140. The direction of fluid flow through second valve 162 is determined by the pressure in the system. In alternative embodiments, one or both of first and second valves 160 and 162 are not incorporated into the system. IV tube 102 and valves 162 can be made of a medical grade plastic and/or rubber.

Cap 144 connects to the top portion of housing 140. Extending out from cap 144 are cap handles 152. Cap handles 152 are sized and shaped to assist a user in compressing piston 142 into its first position, as seen in FIG. 2A. Cap 144 has at least one opening therein to allow portions of piston 142 and locking mechanism 150 to extend therethrough. Cap 144 is configured to restrict the movement of piston 142 such that the first end of piston 142 remains within housing 140. Coupled to a bottom surface of cap 144 is a sterility liner 164. Sterility liner 164 also couples to piston 142 (described below). Sterility liner 164 can be coupled to cap 144 and piston 142 by any suitable means, such as clamps, O-rings, glue, and the like. Sterility liner 164 functions to prevent the interior of housing 140 from becoming contaminated. When piston 142 is in the position illustrated in FIG. 2A, a portion of the interior of housing 140 is exposed. To prevent the interior walls of housing 140 from becoming contaminated, Sterility liner 164 is used to enclose at least a portion of the interior of housing 140 so as to prevent contamination of the interior walls of housing 140. Without sterility linter 164, housing 140 could become contaminated, which contamination would enter the fluid sampling system when piston 142 is moved to the position illustrated in FIG. 2B. Such contamination could expose a risk to a patient. Sterility liner 164 can be made of an air-impermeable material, such as a film of plastic.

A first end of piston 142 is slidably disposed within housing 140. Extending around the edge of the first end of piston 142 is a seal 148. Seal 148 abuts the interior wall of housing 140, creating an airtight seal between piston 142 and housing 140 such that movement of piston 142 within housing 140 causes an increase or decrease in pressure within housing 140. Seal 148 can be an integral part of piston 142 or it can be a distinct piece adapted to be coupled to the first end of piston 142. Seal 148 can be made of a medical grade plastic and/or rubber.

Piston 142 has a rod 154 that extends from the first end of piston 142 and through cap 144. Spring 146 is concentrically placed around rod 154. The first end of spring 146 rests on the top of cap 144. Rod 154 has a rim 156 that extends over the second end of spring 146, thus retaining spring 146 between cap 144 and rim 156. When piston 142 is moved from a second position (shown in FIG. 2B) to the first position shown in FIG. 2, spring 146 is compressed between rim 156 and cap 144. Spring 146 therefore biases piston 142 toward the second position shown in FIG. 2B relative to the first position shown in FIG. 2A.

In addition to rod 154, extending from the first end of piston 142 is locking mechanism 150. In the example embodiment, locking mechanism 150 comprises two levers 150a, 150b that extend from opposing sides of the first end of piston 142 through cap 144. Levers 150a and 150b are biased toward housing 140. Each of levers 150a and 150b has a notch 158 in an outer surface thereof that is sized and shaped to engage cap 144, thus preventing movement of piston 142 relative to housing 140. Levers 150a and 150b can be compressed toward rod 154 to disengage notches 158 from cap 144, thus allowing piston 142 to move relative to housing 140. When cap 144 engages notches 158, piston 142 is held in the position shown in FIG. 2A. As can be seen, even when spring 146 is compressed and locking mechanism 150 is preventing movement of piston 142, there is sufficient space between housing 140 and the first end of piston 142 to allow fluid to flow therethrough.

In use, fluid drawing device 100 is connected to IV tube 102 and piston 142 is locked in the down position illustrated in FIG. 2A. To draw fluid using the present exemplary embodiment of fluid drawing device 100, a user activates fluid drawing device 100 by compressing levers 150a and 150b to disengage piston 142 from housing 140. When piston 142 is disengaged from housing 140, spring 146 biases piston 142 upward to the position illustrated in FIG. 2B. As piston 142 moves upward within housing 140, a negative pressure is created in housing 140 causing first valve 160 to close, preventing fluid from flowing from the first portion of IV tube 102 into housing 140. The negative pressure created in housing 140 when piston 142 moves upward draws the fluid in the second portion of IV tube 102 back into housing 140. Spring 146 is designed to have sufficient travel and force to provide the necessary displacement to draw fluid, such as blood, from a patient into the second portion of IV tube 102. As the fluid in the second portion of IV tube 102 is drawn back into housing 140, a negative pressure is created in the second portion of IV tube 102, which in turn draws fluid from the patient's body back into the second portion of IV tube 102 past sample port 126. After the bodily fluid has been drawn past sample port 126, the user can then insert the fluid sampling device (described in detail below) into sample port 126 to retrieve the desired fluid sample. The negative pressure in housing 140 does not exceed that which is produced during regular phlebotomy by commonly used vacuum packed test tubes. After the fluid sample has been retrieved, the user can return the system to it original configuration (as seen in FIG. 2A) by pressing piston 142 back to its original position which re-infuses the patient's fluid into their body. When piston 142 is depressed, locking mechanism 150 can be re-engaged and fluid flow through the system can be restored to its original rate.

The various components of the fluid drawing device can be made from a medical device industry standard, including, but not limited to thermoplastics, such as Polyethylene (PE), High Density Polyethylene (HDPE), Polypropylene (PP), Polystyrene (PF), Polyethylene Terephthalate (PET), and acrylic (for elements that are desired to be transparent, such as chambers 106, 110 and housing 140, for example), because of their low cost production, ability to be easily molded, sterility, and strength. Spring 146 can be made of steel or any other resilient metal or plastic material. It will be understood by one of ordinary skill in the art in view of the disclosure provided herein that spring 146 can be a flexible member used to store mechanical energy.

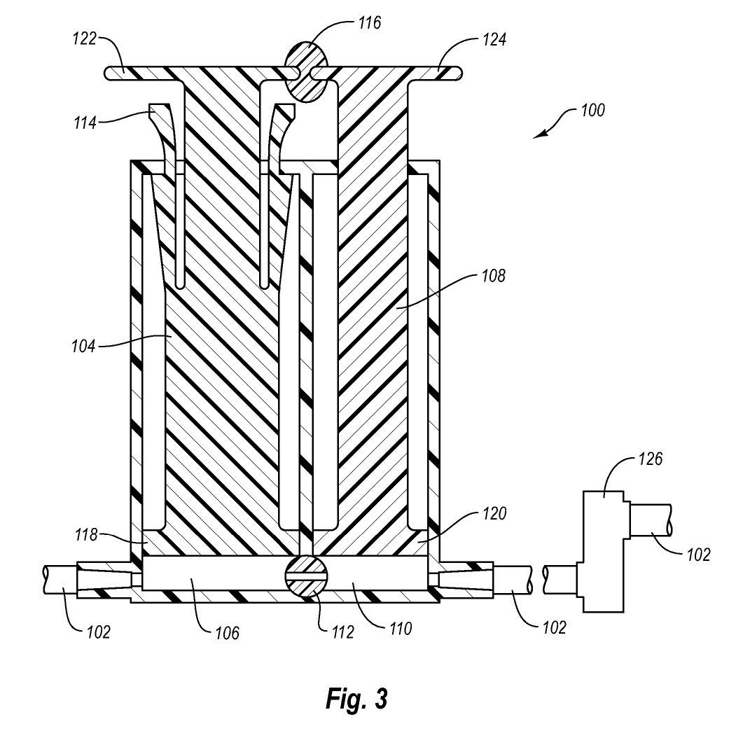

FIG. 3 illustrates an alternative exemplary embodiment of the fluid drawing device 100. The fluid drawing device 100 comprises a driver plunger 104, the first end of which is disposed within a driver chamber 106. The first end of driver plunger 104 has a driver ridge 118 that sealingly engages the interior wall of driver chamber 106. Driver ridge 118 can be an integral part of driver plunger 104 or it can be a distinct piece adapted to couple to the first end of driver plunger 104. In either configuration, driver ridge 118 is adapted to create an airtight seal between driver plunger 104 and driver chamber 106 such that movement of driver plunger 104 within driver chamber 106 causes a corresponding change in pressure within driver chamber 106. Driver ridge 118 can be made of a medical grade plastic or rubber.

The second end of driver plunger 104 comprises a driver handle 122 which extends outside of driver chamber 106. The second end of driver plunger 104 also has a locking mechanism 114 disposed thereon. Locking mechanism 114 comprises at least one leaf spring type structure that extends away from a side of driver plunger 104 and that can engage an interior surface of driver chamber 106 to prevent driver plunger 104 from moving relative to driver chamber 106. Locking mechanism 114 can be compressed to disengage driver plunger 104 from driver chamber 106, thus allowing driver plunger 104 to move relative to driver chamber 106. When locking mechanism 114 is engaged with driver chamber 106, a space between the bottom of driver chamber 106 and the bottom surface of driver plunger 104 is created that is sufficient to allow fluid to flow therethrough.

Fluid drawing device 100 also has a fluid fill plunger 108, the first end of which is disposed within a fluid fill chamber 110. The first end of fluid fill plunger 108 has a ridge 120 that sealingly engages the interior wall of fluid fill chamber 110. Ridge 120 can be an integral part of fluid fill plunger 108 or it can be a distinct piece adapted to couple to the first end of fluid fill plunger 108. In either configuration, ridge 120 is adapted to create an airtight seal between fluid fill plunger 108 and fluid fill chamber 110 such that movement of fluid fill plunger 108 within fluid fill chamber 110 causes a corresponding change in pressure within fluid fill chamber 110. Ridge 120 can be made of a medical grade plastic or rubber.

The second end of fluid fill plunger 108 comprises a fluid fill plunger handle 124 which extends outside of fluid fill chamber 110. Driver plunger handle 122 and fluid fill plunger handle 124 can be connected with connector lock 116 so that the movement of driver plunger 104 and fluid fill plunger 108 is connected. When locking mechanism 114 is engaged with driver chamber 106, thus restricting movement of fluid fill plunger 108 through connector lock 116, a space between the bottom of fluid fill chamber 110 and the bottom surface of fluid fill plunger 108 is created that is sufficient to allow fluid to flow therethrough.

Driver chamber 106 and fluid fill chamber 110 are in fluid communication with each other and with IV tube 102. As can be seen in FIG. 3, a fluid in IV tube 102 can flow through driver chamber 106, into fluid fill chamber 110, and on into a second portion of IV tube 102. The second portion of IV tube 102 has a sample port 126 into which a user may insert a fluid sampling device 200 or 400 to retrieve a fluid sample, such as a blood sample. As the fluid flows from driver chamber 106 to fluid fill chamber 110, it passes through flow gauge 112. Flow gauge 112 is a valve that regulates the amount of fluid that can pass from driver chamber 106 to fluid fill chamber 110, and thus into the patient. Flow gauge 112 can be adjusted by a user to allow a desired amount of fluid to pass from driver chamber 106 to fluid fill chamber 110. In the example embodiment, flow gauge 112 is configured to allow about 3 cc/hr to flow from driver chamber 104 to fluid fill chamber 110. Flow gauge 112 can also acts as a one-way valve to prevent fluid from flowing from fluid fill chamber 110 to driver chamber 106.

As noted above, fluid drawing device 100 can be connected inline with IV tube 102 which can be used for delivering a fluid, such as saline and/or a medicament to a patient. While fluid is being delivered to a patient through IV tube 102, driver plunger 104 and fluid fill plunger 108 of fluid drawing device 100 are in the positions illustrated in FIG. 3. To retrieve a sample of bodily fluid using fluid drawing device 100, a user activates fluid drawing device 100 by compresses locking mechanism 114 which disengages driver plunger 104 from driver chamber 106. The fluid pressure in IV tube 102 causes driver plunger 104 to move up in driver chamber 106. Upward movement of driver plunger 104 reduces the pressure in driver chamber 106 causing flow gauge 112 to close, preventing fluid from flowing from driver chamber 106 to fluid fill chamber 110. Connector lock 116 causes fluid fill plunger 108 to move up simultaneously with driver plunger 104. As fluid fill plunger 108 moves up in fluid fill chamber 110, a negative pressure is created in fluid fill chamber 110 which draws the fluid in the second portion of IV tube 102 back into fluid fill chamber 110. As the fluid in the second portion of IV tube 102 is drawn back into fluid fill chamber 110, a negative pressure is created in the second portion of IV tube 102, which in turn draws a bodily fluid, such as blood, from the patient back into the second portion of IV tube 102 past sample port 126.

After the bodily fluid has been drawn into IV tube 102, the user can then insert fluid sampling device 200 or 400 (described in detail below) to retrieve the desired fluid sample. The negative pressure in fluid fill chamber 110 does not exceed that which is produced during regular phlebotomy by commonly used vacuum packed test tubes. Once the desired fluid sample is retrieved, fluid sampling device 200 or 400 can be removed from sample port 126. The user can then return the system to its original configuration by pressing fluid fill plunger 108 back to its original position which re-infuses the patient's bodily fluid into their body. The user can then press the driver plunger 104 back to its original position and re-engage locking mechanism 114. Once both plungers 104 and 108 are depressed, flow gauge 112 opens and allows the system to deliver the desired amount of fluid to the patient.

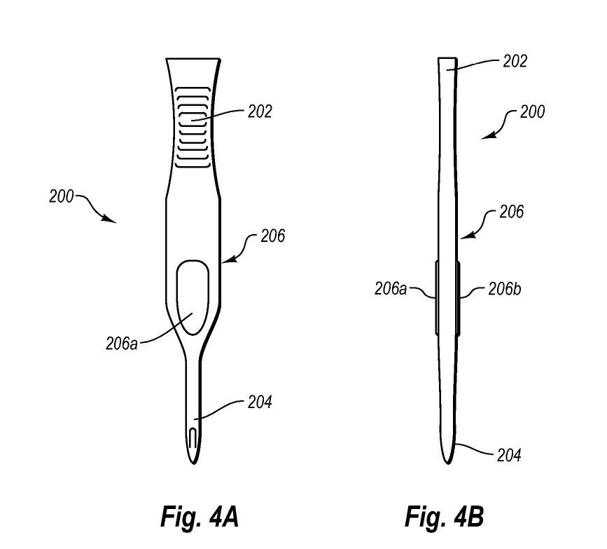

FIGS. 4A and 4B illustrate an exemplary embodiment of the fluid sampling device 200. Fluid sampling device 200 comprises a handle 202, a blunt canula 204, and a main body 206 having an interior portion and windows 206a and 206b. Blunt canula 204 is sized and shaped to fit within sample port 126 (see FIG. 1). Blunt canula 204 has an opening in an end thereof that is in fluid communication with the interior portion of main body 206. The interior portion of main body 206 is adapted to hold a fluid sample, such as a blood sample. Windows 206a and 206b are disposed on opposing sides of main body 206 and allow a user to view the interior portion of main body 206. Windows 206a and 206b also facilitate analysis of the fluid sample when analyzed by a modified glucometer, as discussed below. Handle 202 is ergonomically shaped to allow a user to easily and comfortably hold fluid sampling device 200 with a thumb and forefinger.

In use, after fluid drawing device 100 has been activated and a fluid has been drawn past sample port 126, blunt canula 204 can be inserted into sample port 126. A small volume of fluid can be drawn through the blunt canula into the interior portion of main body 206 by pressure, such as hydrostatic, hemodynamic, and/or mechanically induced pressure. Windows 206a and 206b enable a user to view the fluid sample and determine when a sufficient sample has been obtained. Fluid sampling device 200 can then be removed from sample port 126 and the fluid sample can be analyzed by a modified glucometer, as discussed below.

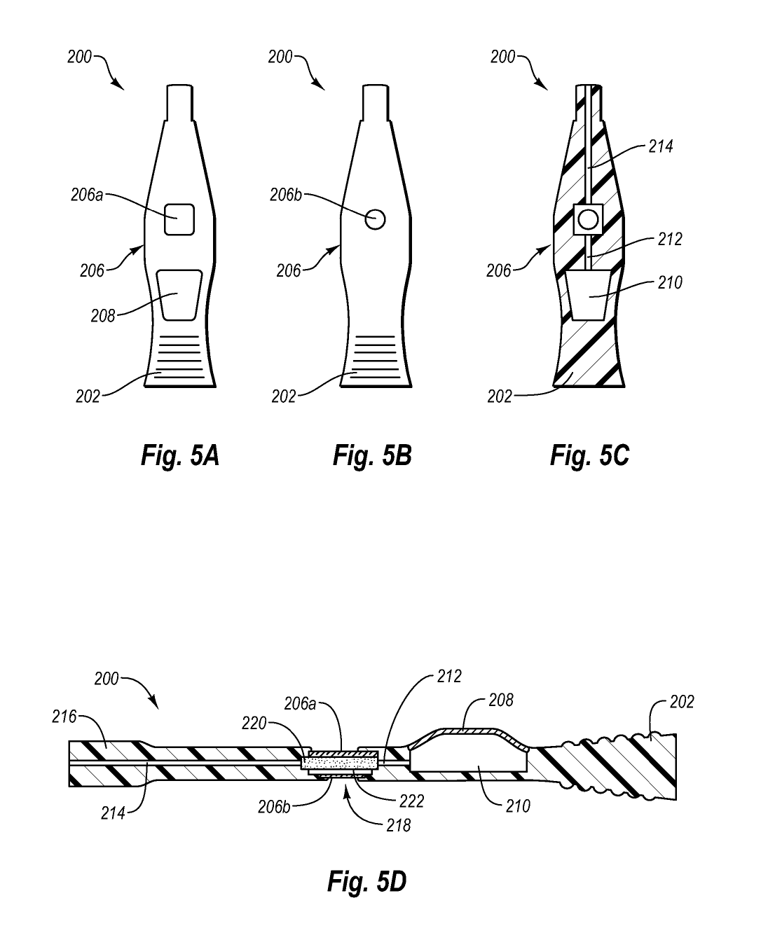

FIGS. 5A-5D illustrate an alternative exemplary embodiment of fluid sampling device 200. Similar to the previous embodiment, fluid sampling device 200 comprises a handle 202, a main body 206, and windows 206a and 206b. In addition, fluid sampling device 200 of the present embodiment comprises a diaphragm 208, a vacuum chamber 210 disposed within main body 206, a first channel 212, a second channel 214, a lure lock tip 216, and a testing compartment 218 disposed within main body 206.

As seen in the Figures, adjacent handle 202, and disposed within main body 206, is a vacuum chamber 210. Vacuum chamber 210 is defined by main body 206 and diaphragm 208. Diaphragm 208 is generally arch shaped and is made from a resilient, pliable material, such as rubber or plastic, so that a user can depress diaphragm 208 to decrease the volume of vacuum chamber 210. Vacuum chamber 210 is in fluid communication with first channel 212 such that when diaphragm 208 is depressed, air from vacuum chamber 210 is expelled through first channel 212. First channel 212 extends from vacuum chamber 210 to testing compartment 218.

Testing compartment 218 is disposed within main body 206 and is partially defined by windows 206a and 206b which are on opposing sides of main body 206. Windows 206a and 206b allow a user to view the interior of testing compartment 218 and determine when a sufficient sample of fluid has been obtained. Within testing compartment 218 is an absorbent material 220, such as foam, and a testing reagent 222. Absorbent material 220 absorbs and retains a fluid sample that is retrieved from sample port 126 as described below. Testing reagent 222 is disposed adjacent absorbent material 220 such that absorbed fluid will contact testing reagent 222. Testing reagent 222 will react to various attributes of the fluid sample, such as glucose levels of a blood sample.

Testing compartment 218 is in fluid communication with second channel 214. Second channel 214 extends through lure lock tip 216 and opens at the end of lure lock tip 216. Lure lock tip 216 can be retrofitted with a blunt canula in order to access various applicable systems, such as sample port 126.

Fluid sampling device 200 of the present embodiment is used in a manner similar to fluid sampling device 200 of the previous embodiment. When fluid drawing device 100 has been activated and a fluid has been draw into IV tube 102 past sample port 126, fluid sampling device 200 can be used to retrieve a fluid sample from sample port 126. The present example embodiment of fluid sampling device 200 does not rely on hydrostatic or hemodynamic pressure to draw fluid from sample port 126 into fluid sampling device 200. Rather, the present embodiment of fluid sampling device 200 utilizes a negative pressure within vacuum chamber 210 to draw fluid from sample port 126 into fluid sampling device 200. Specifically, prior to inserting lure lock tip 216 into sample port 126, a user will depress diaphragm 208 to expel air out of vacuum chamber 210 through first channel 212, testing compartment 218, and second channel 214, thus creating a potential vacuum within vacuum chamber 210. Once the potential vacuum has been created in vacuum chamber 210, lure lock tip 216, either as shown in the FIGS. 5A-5D or retrofitted with a blunt canula, is inserted into sample port 126 (see FIG. 1). The user then releases diaphragm 208 to return to its original shape and position. As diaphragm 208 returns to its original position, a negative pressure or vacuum is created within vacuum chamber 210. Vacuum chamber 210 and diaphragm 208 are sized adapted to create a negative pressure within fluid sampling device 200 sufficient to draw a desired quantity of fluid from sample port 126, through second channel 214, and into testing compartment 218.

As the fluid enters testing compartment 218, absorbent material 220 will absorb the fluid and distribute it across testing reagent 222. Absorbent material 220 also functions to retain fluid within fluid sampling device 200. Windows 206a and 206b allow a user to view absorbent material 220 in testing compartment 218 to determine when an adequate fluid sample has been achieved. When a sufficient quantity of fluid has been drawn into testing compartment 218, the user removes fluid sampling device 200 from sample port 126.

Having obtained a fluid sample within fluid sampling device 200, the fluid sample can then be analyzed using a modified glucometer 300 as seen in FIG. 7. Glucometers are well known in the art. A typical glucometer 300 comprises a housing 302, a keypad 304, internal analysis apparatus 306 (not shown), and a receptacle 308 for receiving a test strip having a fluid sample, such as a blood sample, disposed thereon. The modified glucometer shown in FIG. 7 has a reconfigured receptacle 308 that is designed to receive fluid sampling device 200 therein. Receptacle 308 is adapted such that when fluid sampling device 200 is inserted therein, windows 206a and 206b are in alignment with an analyzer light (not shown) of modified glucometer 300. The analyzer light of modified glucometer 300 is directed through one or both of windows 206a and 206b to analyze various attributes of the fluid sample, such as glucose levels of a blood sample.

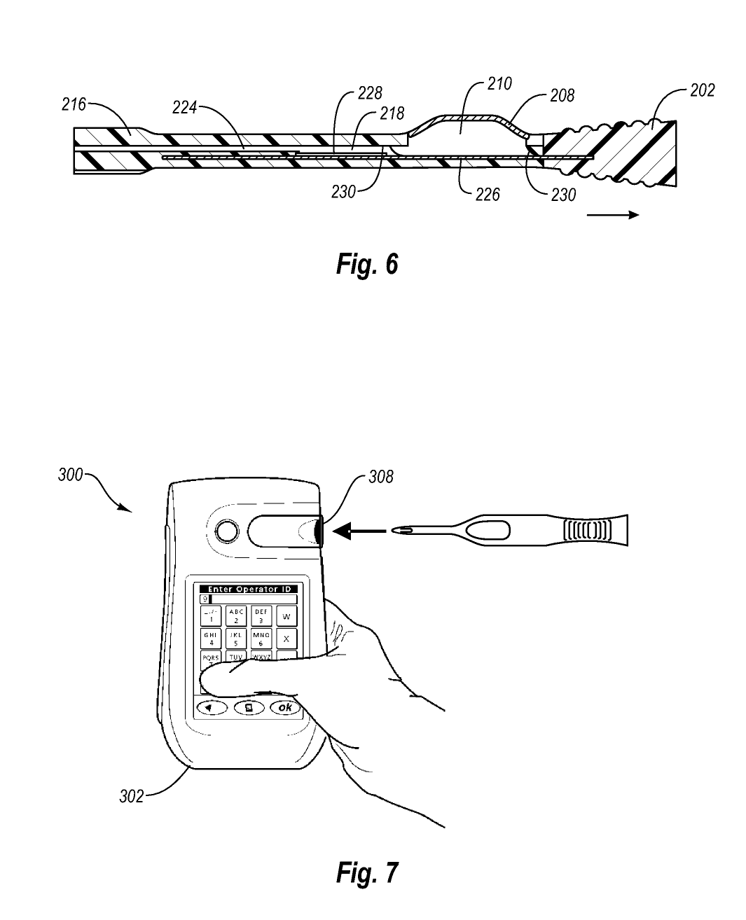

FIG. 6 illustrate yet another exemplary embodiment of fluid sampling device 200. Fluid sampling device 200 of the present embodiment comprises handle 202, vacuum chamber 210, diaphragm 208, testing compartment 218, lure lock tip 216, channel 224, test strip 226, reagent 228, and valves 230. Vacuum chamber 210 is in fluid communication with channel 224 to allow air to be expelled from vacuum chamber 210 through channel 224 when diaphragm 208 is depressed. Expulsion of air from vacuum chamber 210 creates a potential vacuum therein.

Fluid sampling device 200 is adapted to receive a standard test strip 226 disposed at least partially therein. In the illustrated embodiment, test strip 226 is received within testing compartment 218, vacuum chamber 210 and handle 202. Fluid sampling device 200 can be adapted to receive standard supply test strips such as the SureStep Pro test strip made by LifeScan (a J&J subsidiary).

Fluid sampling device 200 of the present embodiment is used in a manner similar to the previous embodiments of fluid sampling device 200 equipped with diaphragm 208. Specifically, a user depresses diaphragm 208 to expel air out of vacuum chamber 210 and create a potential vacuum therein. Lure lock tip 216, either as illustrated or retrofitted with a blunt canula, is inserted into sample port 126 and diaphragm 208 is released to create a negative pressure within vacuum chamber 210. The negative pressure within vacuum chamber 210 draws fluid from sample port 126 through channel 224 into testing compartment 218. Fluid entering testing compartment 218 is absorbed by test strip 226. Having received a sufficient fluid sample, fluid sampling device 200 can be removed from sample port 126.

As seen in FIG. 6, unlike the previous embodiments of fluid sampling device 200, handle 200 of the present embodiment can be removed to expose an end of test strip 226. Handle 200 can be coupled to fluid sampling device 200 with the use of clamps, clips, or the like. With handle 202 detached from fluid sampling device 200, test strip 226 can be removed from fluid sampling device 200 for analysis of the fluid sample. By adapting fluid sampling device 200 with a removable handle 202, the fluid sample disposed on test strip 226 can be analyzed using conventional means, such as a typical glucometer that is adapted to receive standard test strips. To prevent potential air leaks through handle 202 that would reduce the drawing force of vacuum chamber 210 and diaphragm 208, fluid sampling device 200 has one-way valve 230 disposed across the opening through which test strip 226 is inserted.

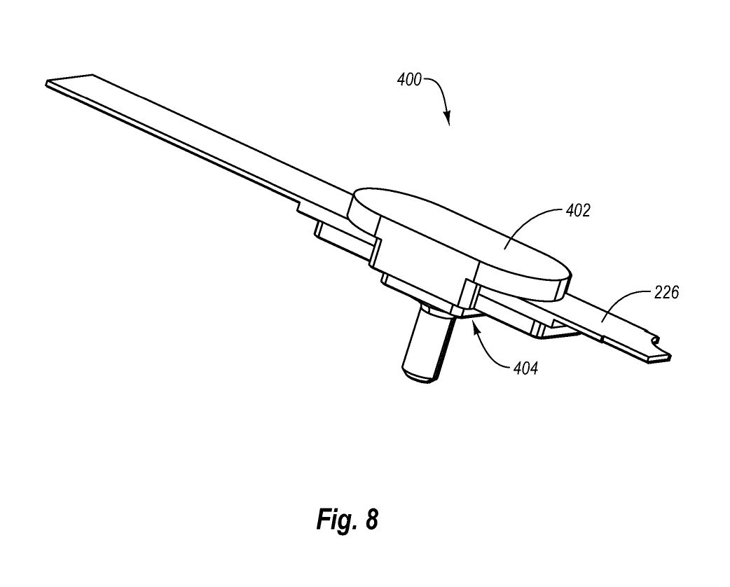

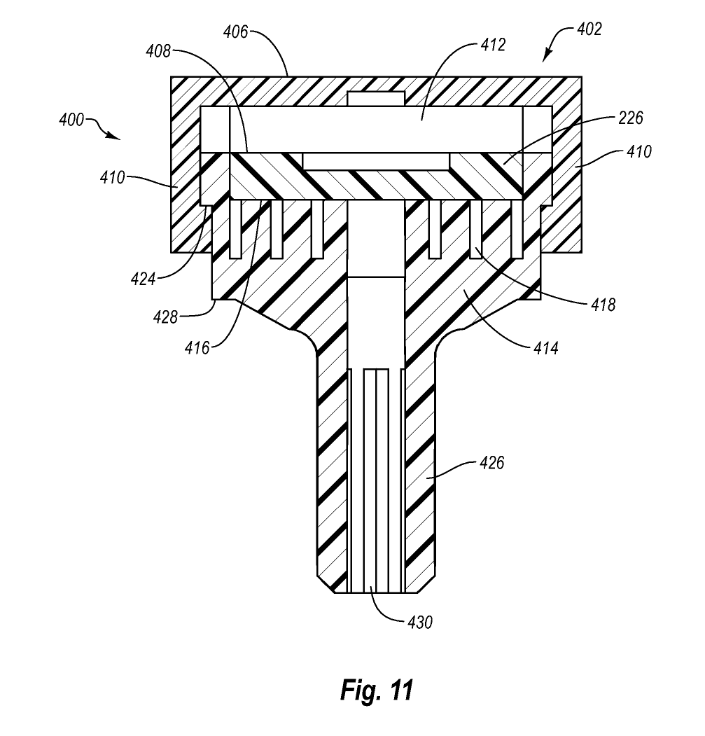

FIG. 8 illustrates a perspective view of yet another embodiment of fluid sampling device 400. Fluid sampling device 400 of the present embodiment comprises a top portion 402 and a base portion 404 that are adapted to be coupled together. Top portion 402 and base portion 404 can be couple together in a first position and a second position. In the first position, fluid sampling device 400 can have a test strip 226 disposed between base portion 404 and top portion 402 (as seen in FIGS. 8 and 11). It is contemplated that standard test strips, such as the SureStep Pro test strip made by LifeScan (a J&J subsidiary) can be used in combination with fluid sampling device 400. Top portion 402 is biased toward base portion 404 such that when test strip 226 is removed top portion 402 moves toward base portion 404. Fluid sampling device 400 can be configured to be inserted within sample port 126 to obtain a fluid sample as illustrated in FIG. 1. In an alternative embodiment, top portion 402 can comprise test strip 226. In this embodiment, base portion 404 and test strip 226 can be coupled together with a flange, and adhesive such as glue, or a mechanical fastener.

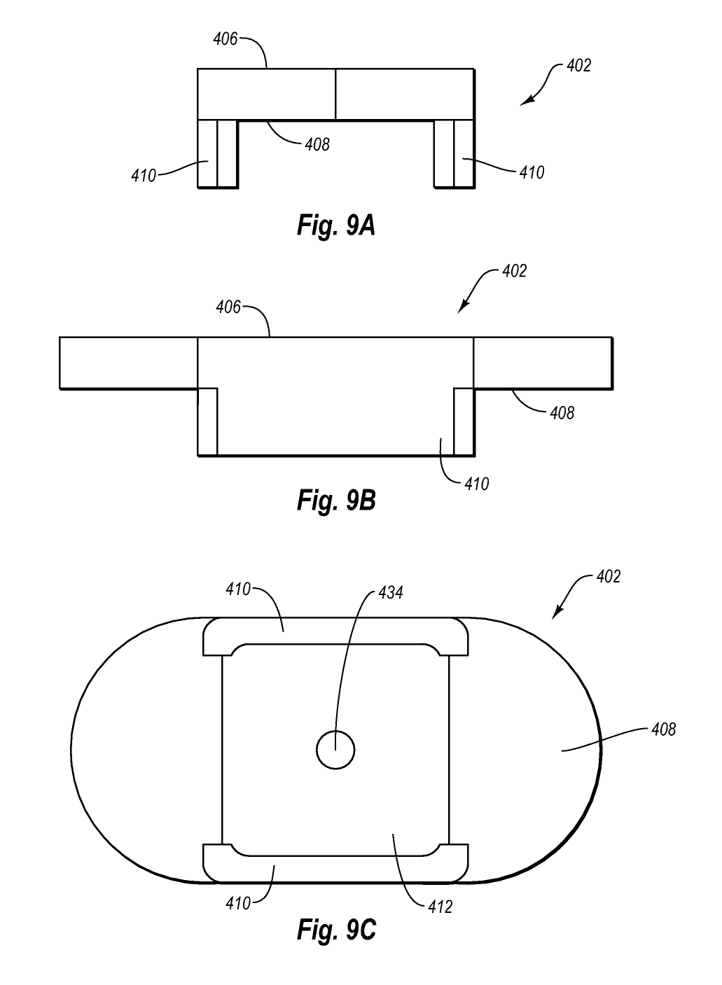

FIGS. 9A-9C illustrate end, side, and bottom views of top portion 402. As seen in the Figures, top portion 402 comprises a top surface 406, a bottom surface 408, flanges 410, and recess 412. Top surface 402 defines an aperture 434 that can allow a user to view the interior of fluid sampling device 400 to determine when a sufficient fluid sample had been obtained. Flanges 410 are disposed on opposing sides of top portion 402 and extend from bottom surface 408. The ends of flanges 410 are rounded in toward the center of top portion 402. Recess 412 is a cavity in bottom surface 408. The various elements of top portion 402 can be integrally formed as a unitary piece, or the elements can be individually formed and then coupled together. As noted above, top portion 402 can comprise test strip 226.

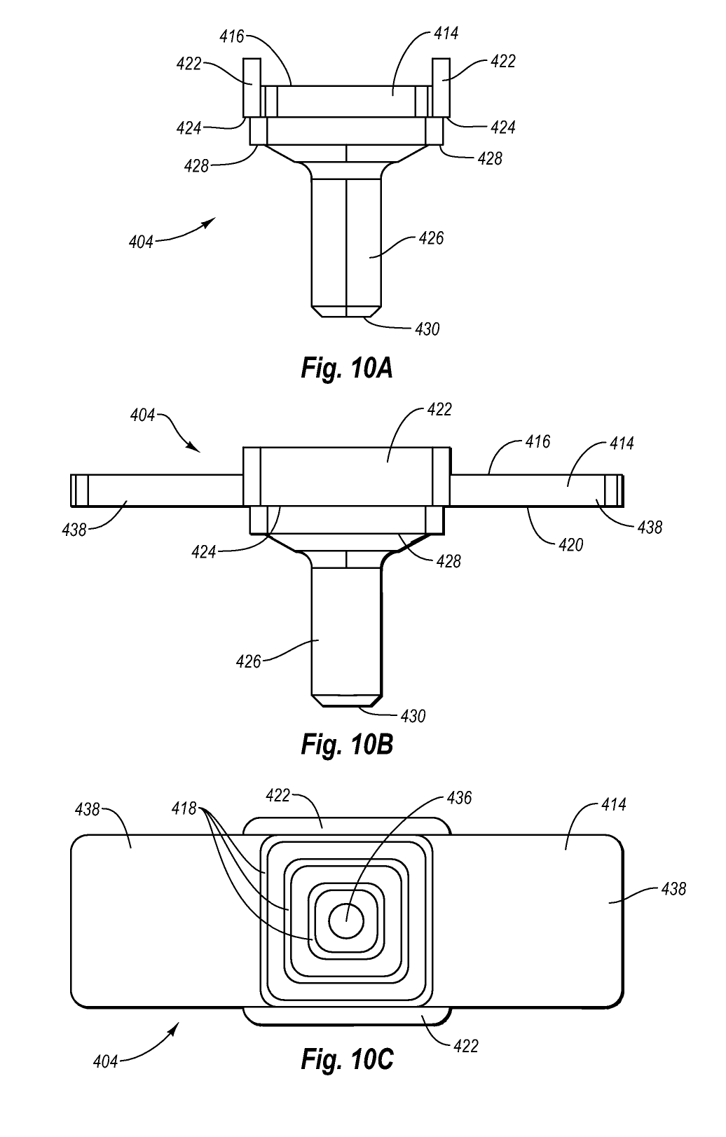

FIGS. 10A-10C illustrate end, side, and bottom views of base portion 404. Base portion 404 comprises a platform 414, walls 422, and a blunt canula 426. Platform 414 has a bottom surface 420 and a top surface 416 with grooves 418 therein as seen in FIG. 10C. The opposing ends 438 of platform 414 can function as handles to facilitate simple and convenient use of fluid sampling device 400. Walls 422 are disposed on opposing sides of platform 414. The ends of wall 422 are rounded in toward the center of base portion 404. Walls 422 have first ridges 424 and second ridges 428 extending along its outer surface. As seen best in FIG. 10A, first ridges 424 are vertically above second ridges 428 and extend further out than second ridges 428. Extending from bottom surface 420 is blunt canula 426. Blunt canula 426 has a lumen 430 extending from a distal end of blunt canula 426 through an aperture 436 in platform 414. The distal end of blunt canula 426 is adapted to be inserted into sample port 126 to obtain a fluid sample, such as a blood sample.

FIG. 11 is a cross-sectional end view of fluid sampling device 400 illustrating how top portion 402 and base portion 404 couple together. As noted above, the ends of wall 422 and flanges 410 are rounded. The rounded ends of walls 422 and flanges 410 facilitate alignment of top portion 402 and base portion 404 and prevent horizontal movement of top portion 402 relative to base portion 404. The interior surfaces of flanges 410 each has an inwardly projecting ridge 432 that is adapted to engage ridges 424 and 428. Specifically, top portion 402 is aligned with base portion 404 and flanges 410 extend over walls 422 and ridges 432 engage ridges 424 when test strip 226 is disposed between top portion 402 and base portion 404. Top portion 402 is sized such that when test strip 226 is in place and ridges 432 are engaged with ridges 424, flanges 410 are deflected slightly away from base portion 404. When test strip 226 is removed, the deflection in flanges 410 causes top portion 402 to be biased toward base portion 404. Therefore, when test strip 226 is removed, top portion 402 moves toward base portion 404 until bottom surface 408 of top portion 402 comes into contact with top surface 416 of platform 414. At this point, ridges 432 of flanges 410 engage ridges 428 of walls 422. The engagement between ridges 432 and either ridges 424 or ridges 428 prevents top portion 402 from becoming undesirably separated from base portion 404. In embodiments in which top portion 402 comprises test strip 226, test strip 226 can be couple to base portion 404 with a flange similar to flanges 410, or test strip 226 can be bonded to base portion with a glue or plastic.

In use, fluid sampling device 400 will have test strip 226 inserted between top portion 402 and base portion 404. After fluid drawing device 100 has been activated and a fluid has been drawn into IV tube 102 past sample port 126, a user can insert the distal end of blunt canula 426 into sample port 126. Pressure, such as hydrostatic, hemodynamic, or mechanically induced pressure, causes fluid from sample port 126 to enter lumen 430 and move up through base portion 404 and onto test strip 226. When a sufficient fluid sample has been obtained, fluid sampling device 400 can be removed from sample port 126. Test strip 226 can then be withdrawn from between top portion 402 and base portion 404 and analyzed in a glucometer as described above. In embodiments in which test strip 226 comprises top portion 402, fluid sampling device 400 can be removed from sample port 126 after a fluid sample has been obtained and a glucometer modified to receive fluid sampling device 400 can be used to analyze the fluid sample. In some embodiments in which test strip 226 comprises top portion 402, test strip 226 can be removed from base portion 404 and the fluid sample can be analyzed with a standard glucometer.

When fluid, such as blood, is drawn from sample port 126 into fluid sampling device 400, excess fluid may be received within fluid sampling device 400 and on test strip 226. To prevent excess fluid from leaking out of fluid sampling device 400 or remaining on test strip 226 when it is removed from fluid sampling device 400, top portion 402 has recess 412 and base portion 404 has grooves 418 in top surface 416 that cooperate to eliminate these problems. Specifically, grooves 418 act like a squeegee to remove excess fluid from test strip 226 as test strip 226 is withdrawn from fluid sampling device 400. When test strip 226 is removed from fluid sampling device 400, top portion 402 and base portion 404 come together as described above, and grooves 418 and recess 412 cooperate to retain the excess fluid within fluid sampling device 400.

As with fluid drawing device 100, fluid sampling device 200 and fluid sampling device 400 can be made from medical device industry standard plastics including, but not limited to thermoplastics, such as Polyethylene (PE), High Density Polyethylene (HDPE), Polypropylene (PP), Polystyrene (PF), Polyethylene Terephthalate (PET), and acrylic (for transparent properties), because of their low cost production, ability to be easily molded, sterility, and strength.

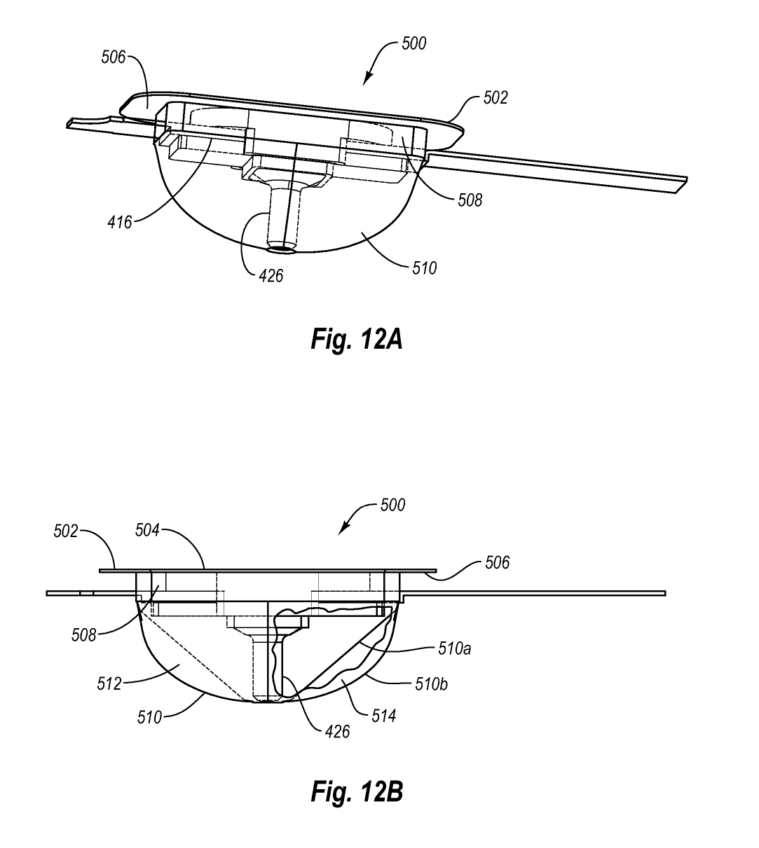

FIGS. 12A-12B illustrate an example embodiment of a protective housing 500 that is adapted to receive fluid sampling device 400 therein. Protective housing 500 comprises substrate 502, a railing 508, and a cover portion 510. Substrate 502 has a top surface 504 and a bottom surface 506 that lays adjacent top surface 406 of top portion 402. Extending from bottom surface 506 is railing 508. Railing 508 extends around fluid sampling device 400 and down to about top surface 416 of platform 414. Substrate 502 and railing 508 can be formed as a unitary piece, or they can be individually formed and coupled together.

Attached to the lower edge of railing 508 is cover portion 510. Cover portion 510 extends over blunt canula 426. Cover portion 510 can comprise two layers of plastic 510a and 510b with air disposed therebetween. Layers of plastic 510a and 510b can be bonded together at various points to create pockets of air therein. As shown in FIG. 12B, layers of plastic 510a and 510b are bonded together near railing 508 and the distal end of blunt canula 426, thus creating air pockets 512 and 514. In some embodiments, cover portion 510 comprises a single layer of plastic 510a that extends from railing 508 around base portion 404.

Protective housing 500 is adapted to protect fluid sampling device 400 from physical damages prior to use, such as during shipment. Protective housing 500 also maintains the sterility of fluid sampling device 400 by preventing undesirable contamination through exposure to a non-sterile surface, such as a user's hands.

Fluid sampling device 400 packaged in protective housing 500 is used in a manner similar to that described above with respect to fluid sampling device 400. However, when fluid sampling device 400 is packaged in protective housing 500, cover portion 510 must be ruptured prior to inserting blunt canula 426 into sample port 126. Typically, a user will hold fluid sampling device 400, disposed within protective packaging 500, with their thumb or palm on top surface 504 of substrate 502, and their index and middle fingers extending around opposing sides of cover portion 510. Holding fluid sampling device 400 and protective housing 500 in this manner, a user can squeeze protective housing 500 with enough force to rupture air pockets 512 and 514. Once air pockets 512 and 514 have been ruptured, the distal end of blunt canula 426 can be forced through layers of plastic 510a and 510b to expose blunt canula 426. With blunt canula 426 exposed, a user can then insert blunt canula into sample port 126 to obtain a fluid sample in the same manner as previously described.

In one embodiment, the fluid drawing device can comprise a housing having a fill chamber in fluid communication with a patient injection site. A piston can be positionable within the fill chamber to sealingly engage the fill chamber, wherein the piston has a first position and a second position, wherein movement of the piston from the first position to the second position generates a negative pressure in the fill chamber effective to draw fluid from the patient injection site into the fill chamber. The fluid drawing device can also include biasing means for biasing the piston to the second position relative to the first position. The biasing means can comprise a spring linked to the piston, such that the spring is more compressed when the piston is in the first position than when the piston is in the second position. In some embodiments, the biasing means is generated by fluid pressure from the patient injection site. In other embodiments, the biasing means is generated by hydraulic actuation. The fluid drawing device can also include securing means for selectively securing the piston in the second position. The securing means can comprise a locking system at least partially coupled to the piston.

In an alternative exemplary embodiment, the fluid drawing device can comprise a housing having a fill chamber in fluid communication with a patient injection site, a piston disposed and positionable within the fill chamber, the piston being sealingly engaged with the fill chamber to generate a negative pressure in the fill chamber effective to draw fluid from the patient injection site into the fill chamber when the piston is moved from a first position to a second position. The fluid drawing device can also include biasing means for biasing the piston to the second position relative to the first position, and securing means for selectively securing the piston in the first position. The biasing means of the fluid drawing device can comprise a spring linked to the piston, such that the spring is more compressed when the piston is in the first position than when the piston is in the second position. The biasing means can also be generated by fluid pressure from the patient injection site. The biasing means can also be generated by hydraulic actuation. The securing means can comprise a locking system at least partially coupled to the piston.

In another alternative example embodiment, the fluid drawing device can comprise a housing having a fill chamber in fluid communication with a patient injection site, a piston positionable within the fill chamber to sealingly engage the fill chamber, wherein the piston has a first position and a second position, wherein movement of the piston from the first position to the second position generates a negative pressure in the fill chamber effective to draw fluid from the patient injection site into the fill chamber, a spring linked to the piston, wherein the spring is more compressed when the piston is in the first position than when the spring is in the second position, such that the spring tends to bias the piston to the second position relative to the first position; and a locking member coupled to the piston to selectively secure the piston in the first position. The locking member can be positionable to engage the housing to secure the piston in the first position. The locking member can be configured to be selectively disengaged from the housing so as to enable the piston to move between the first and the second positions.

One embodiment, a method of sampling bodily fluid comprises: providing a fluid sampling system, wherein said fluid sampling system comprises an IV tube in fluid communication with a patient injection site, said IV tube having a sample port; a fluid drawing device in fluid communication with said IV tube, said fluid drawing device having a fill chamber and a piston sealingly engaged therein; and a fluid sampling device that is configured to be inserted within said sample port, said fluid sampling device having a blunt canula; drawing a fluid through said IV tube to sample port by activating fluid drawing device, wherein activation of said fluid drawing device comprises moving said piston relative to said fill chamber such that a negative pressure is generated in fluid drawing device sufficient to draw a fluid through IV tube to said sample port; and obtaining a fluid sample from said sample port by inserting said blunt canula into said sample port and drawing fluid into said fluid sampling device by hydrostatic or hemodynamic pressure, or negative pressure.

In some embodiments of the method, the fluid drawing device comprises two fill chambers and two pistons. In some embodiments, the fluid drawing device further comprises a handle, a main body having an interior portion, and a window in the main body. In some embodiments the fluid drawing device is adapted to receive a test strip therein. In some embodiments the handle of the fluid drawing device is removable to facilitate removal of the test strip from the fluid drawing device. The fluid sampling device has an absorbent material disposed in the interior portion in some embodiments.

In other embodiments, the fluid sampling device further comprises a base portion having a platform and a ridge extending around at least a portion of an outer surface of the base portion; and a top portion having a flange adapted to engage the ridge to couple the top portion to the base portion. In some embodiments, the fluid sampling device is adapted to receive a test strip between the base portion and the top portion. The fluid sampling device can be configured to allow the test strip to be removed from the fluid sampling device to facilitate analysis of the fluid sample.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A fluid sampling device comprising:
a first portion having a blunt canula, a platform in fluid communication with said blunt canula, and a ridge operatively associated with said platform; and
a second portion having a flange adapted to engage said ridge to couple said second portion to said first portion.

2. The medical device of claim 1, wherein said first portion and said second portion are adapted to be coupled together with a test strip therebetween.

3. The medical device of claim 1, wherein said second portion is adapted to couple to said first portion in a first position and in a second position relative to said first portion, said second portion adapted to be biased toward said second position.

4. The medical device of claim 3, wherein said second portion is in said first position when a test strip is placed between said second portion and said first portion.

5. The medical device of claim 4, further comprising a housing that is adapted to protect said first and second portions from damage and prevent contamination of said blunt canula, said housing comprising:
a substrate that lies adjacent said second portion;
a railing extending from said substrate; and
a cover coupled to said railing and which extends around at least of portion of said first portion.

6. The medical device of claim 5, wherein said second portion moves from said first position to said second position when said test strip is removed from between said first portion and said second portion.

7. The medical device of claim 6, wherein said second portion and said first portion are adapted to remove and retain excess fluid from said test strip when said test strip is removed from between said first portion and said second portion.

8. A medical device comprising:
a blunt canula adapted to be inserted into a sample port of an intravenous tube, said blunt canula comprising a lumen;
a base portion in fluid communication with said blunt canula, said base portion having an aperture in fluid communication with said lumen of said blunt canula; and
a top portion adapted to be coupled to said base portion, said top portion having an aperture therein that is in fluid communication with said aperture of said base portion and said lumen of said blunt canula.

9. The medical device of claim 8, wherein said top portion and said base portion are adapted to receive a test strip therebetween.

10. The medical device of claim 9, wherein said aperture of said top portion is adapted to allow a user to see a portion of said test strip through said aperture of said top portion to determine when a predetermined amount of fluid has been absorbed by said test strip.

11. The medical device of claim 10, wherein said top portion has a recess in a bottom surface thereof and a top surface of said base portion has at least one groove therein, said recess in said top portion and said at least one groove in said base portion are adapted to remove and retain excess fluid from said test strip when said test strip is removed from between said top portion and said base portion.

12. The medical device of claim 9, wherein said top portion is adapted to be coupled to said base portion in a first position and a second position relative to said base portion, said top portion adapted to be biased toward said second position.

13. The medical device of claim 9, wherein said base portion has at least one handle portion.

14. The medical device of claim 9, further comprising a housing that is adapted to enclose said top and base portions and prevent premature exposure of said blunt canula, said housing comprising:
a substrate that lies adjacent said top portion; and
a cover linked to said substrate and which extends around at least a portion of said base portion.

15. A medical device comprising:
a fluid sampling device adapted to be in fluid communication with a sample port of an intravenous tube, said fluid sampling device comprising
a base portion having a blunt canula extending therefrom; and
a top portion adapted to be coupled to said base portion; and
a test strip adapted to be movably placed between said base portion and said top portion, wherein said fluid sampling device is adapted to transfer fluid from the intravenous tube to said test strip through said blunt canula.

16. The medical device of claim 15, wherein said top portion has at least one recess in a bottom surface thereof and said base portion has at least one recess in a top surface thereof, said at least one recess of each of said top portion and said base portion cooperate to remove and retain excess fluid from said test strip when said test strip is removed from said fluid sampling device.

17. The medical device of claim 15, wherein said base portion of said fluid sampling device has an aperture therein that is in fluid communication with said blunt canula and said test strip.

18. The medical device of claim 15, wherein said top portion of said fluid sampling device has an aperture therein adapted to allow a user to determine when a predetermined amount of fluid has been collected on said test strip.

19. The medical device of claim 15, wherein said top portion of said fluid sampling device can be connected to said base portion of said fluid sampling device in a first position and a second position, said top portion being biased toward said second position.

20. The medical device of claim 15, further comprising a housing that is adapted to enclose said fluid sampling device and at least a portion of said test strip to prevent premature exposure of said blunt canula, said housing comprising:
a substrate that lies adjacent said top portion;
a railing extending from said substrate; and
a cover coupled to said railing and which extends around at least a portion of said base portion.
